(19) United States
(12) Patent Application Publication
(10) Pub. No.: US 2024/0009440 A1
(43) Pub. Date: Jan. 11, 2024

(54) LOCKING FEATURE FOR LVAD CONNECTOR

(71) Applicant: Medtronic, Inc., Minneapolis, MN (US)

(72) Inventors: Kevin R. Seifert, Forest Lake, MN (US); Jonathan A. Hughes, Blaine, MN (US); Lisa A. Dalluge, Faribault, MN (US); Andrew J. Ries, Lino Lakes, MN (US); Thomas J. Fick, Minneapolis, MN (US); Richard P. Nelson, Hudson, WI (US)

(21) Appl. No.: 18/251,424

(22) PCT Filed: Sep. 28, 2021
(86) PCT No.: PCT/US2021/052267

§ 371 Date: May 2, 2023

Related U.S. Application Data
(60) Provisional application No. 63/114,591, filed on Nov. 17, 2020.

(51) Int. Cl. A61M 39/10

(52) U.S. Cl. CPC A61M 39/1011

(57) ABSTRACT

A locking mechanism for a connector to an implantable controller includes a housing of the implantable controller defining a housing bore sized to receive the connector. A set screw is releasably coupled to the housing and disposed at an oblique angle with respect to the housing, the set screw being configured to engage at least a portion of the connector when the set screw is fully inserted within the housing to lock the connector within the housing.

### FIELD

The present technology is generally related to a locking mechanism between a connector for an implantable blood pump and a controller.

### BACKGROUND

Fully implantable blood pump systems include implantable controllers and connectors that engage these controllers. In particular, a first connection is required between an implantable receiving coil of a transcutaneous energy transfer system (TETS) and the implanted controller and second connection is required between the implantable blood pump and the implanted controller. To facilitate such connections, a header of the implantable controller provides access for the connectors to the implantable controller.

### SUMMARY

The techniques of this disclosure generally relate to a locking mechanism between ac connector for an implantable blood pump and a controller.

In one aspect, a locking mechanism for a connector to an implantable controller includes a housing of the implantable controller defining a housing bore sized to receive the connector. A set screw is releasably coupled to the housing and disposed at an oblique angle with respect to the housing, the set screw being configured to engage at least a portion of the connector when the set screw is fully inserted within the housing to lock the connector within the housing.

In another aspect of this embodiment, the set screw is configured to engage a flange of a distal end of the connector when fully inserted within the housing.

In another aspect of this embodiment, the housing bore tapers in diameter as it extends distally.

In another aspect of this embodiment, the set screw includes a proximal portion and a distal portion, and wherein the proximal portion includes a plurality of threads and the distal portion includes an elongate rod.

In another aspect of this embodiment, the elongate rod includes a proximal end and a distal end, and wherein the distal end includes a stop shoulder, and wherein the distal end is configured to engage the flange when the set screw is fully inserted within the housing.

In another aspect of this embodiment, the proximal end of the elongate rod includes a shoulder, and wherein the shoulder is configured to engaged a corresponding depth stop defined by the housing.

In another aspect of this embodiment, the stop shoulder is atraumatic in shape.

In another aspect of this embodiment, the locking mechanism further includes a retainer pin retained within the housing, the retainer pin being configured to prevent the set screw from being removed when the set screw is fully retracted.

In another aspect of this embodiment, the retainer pin is disposed at an angle orthogonal to the set screw.

In another aspect of this embodiment, the retainer pin is disposed at an oblique angle with respect to the housing.

In another aspect of this embodiment, the set screw is configured to engaged to an atraumatic locking element, and wherein the atraumatic locking element is pushed into contact with the connector when then set screw is fully inserted.

In another aspect of this embodiment, the atraumatic locking element is a sphere.

In one aspect, a locking mechanism for a connector to an implantable controller includes a housing of the implantable controller defining a housing bore sized to receive the connector, the housing bore defining a diameter that tapers as it extends distally. A set screw is releasably coupled to the housing and disposed at an oblique angle with respect to the housing, the set screw being configured to engage a flange of the connector when the set screw is fully inserted within the housing to lock the connector within the housing. A retainer pin is releasably retained within the housing, the retainer pin being configured to prevent the set screw from being removed when the set screw is fully retracted.

In another aspect of this embodiment, the set screw includes a proximal portion and a distal portion, and wherein the proximal portion includes a plurality of threads and the distal portion includes an elongate rod.

In another aspect of this embodiment, the elongate rod includes a proximal end and a distal end, and wherein the distal end includes a stop shoulder, and wherein the stop shoulder is configured to engage the flange when the set screw is fully inserted within the housing.

In another aspect of this embodiment, the proximal end of the elongate rod includes a chamfered shoulder, and wherein the chamfered shoulder is configured to engaged a corresponding depth stop defined by the housing.

In another aspect of this embodiment, the stop shoulder is atraumatic in shape.

In another aspect of this embodiment, the retainer pin is disposed at an oblique angle with respect to the housing.

In another aspect of this embodiment, the retainer pin is disposed at an angle orthogonal to the set screw.

In one aspect, a locking mechanism for a connector to an implantable controller including a housing of the implantable controller defining a housing bore sized to receive the connector, the housing bore defining a diameter that tapers as it extends distally. A set screw is releasably coupled to the housing and disposed at an oblique angle with respect to the housing, the set screw being configured to engage a flange of the connector when the set screw is fully inserted within the housing to lock the connector within the housing. The set screw includes a proximal portion and a distal portion, and the proximal portion includes a plurality of threads and the distal portion includes an elongate rod, the elongate rod includes a proximal end and a distal end, and wherein the distal end includes an atraumatic stop shoulder, and wherein the stop shoulder is configured to engage the flange when the set screw is fully inserted within the housing, the proximal end of the elongate rod further includes a chamfered shoulder, and the chamfered shoulder is configured to engaged a corresponding depth stop defined by the housing. A retainer pin is releasably retained within the housing and disposed at an oblique angle with respect to the housing and orthogonally disposed with respect to the set screw, the retainer pin being configured to prevent the set screw from being removed when the set screw is fully retracted.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an internal system view of an implantable blood pump with a TETS receiver source constructed in accordance with the principles of the present application;

FIG. 2A is a side perspective view of an embodiment of a locking mechanism in an unlocked position;

FIG. 2B is a side perspective view of the embodiment of the locking mechanism shown in FIG. 2A in a locked position;

FIG. 3 is a front perspective view of an embodiment of a housing and a connector with a locking mechanism;

FIG. 4 is a front perspective view of another embodiment of a housing and a connector with a locking mechanism;

FIG. 5A is a side perspective view of another embodiment of a locking mechanism in an unlocked position;

FIG. 5B is a side perspective view of the embodiment shown in FIG. 5A with a sphere being pushed by a flange; and

FIG. 5C is a side perspective view of the embodiment shown in FIG. 5A in a locked position.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

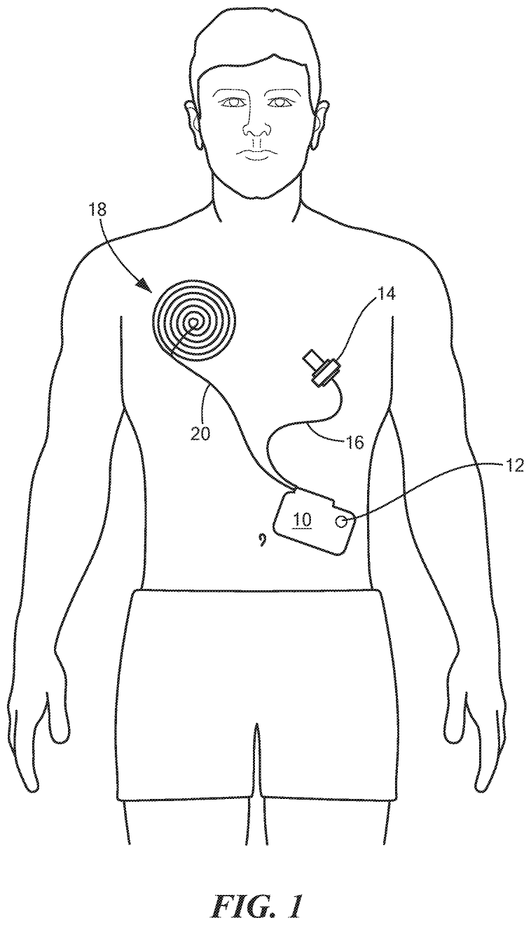

Referring to the drawings in which like reference designators refer to like elements, there is shown in FIG. 1 an exemplary implantable controller for an implantable medical device constructed in accordance with the principles of the present application and designated generally as “10.” The controller 10 may include one or more batteries 12 configured to power the components of the controller and provide power one or more implantable medical device, for example, a blood pump such as ventricular assist device (VAD) 14 implanted within the left ventricle of the patient's heart. VADs 14 may include centrifugal pumps, axial pumps, or other kinds electromagnetic pumps configured to pump blood from the heart to blood vessels to circulate around the body. One such centrifugal pump is the HVAD sold by HeartWare, Inc. and is shown and described in U.S. Pat. No. 7,997,854 the entirety of which is incorporated by reference. One such axial pump is the MVAD sold by HeartWare, Inc. and is shown and described in U.S. Pat. No 8,419,609. In an exemplary configuration, the VAD 14 is electrically coupled to the controller 10 by one or more implanted conductors that form a driveline 16 configured to provide power to the VAD 14, relay one or more measured feedback signals from the VAD 14, and/or provide operating instructions to the VAD 14. The controller 10 may include processing circuity having one or more processors configured to operate the VAD 14 and to processes various signals received from the VAD 14.

Continuing to refer to FIG. 1, a receiving coil 18 may also be coupled to the controller 10 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission coil (not shown) coupled to an external battery and controller (not shown) disposed opposite the receiving coil 18 on the outside of the patient's body. The receiving coil 18 may be disposed within a hermetically sealed package that does not interfere with the conductivity of the receiving coil 18.

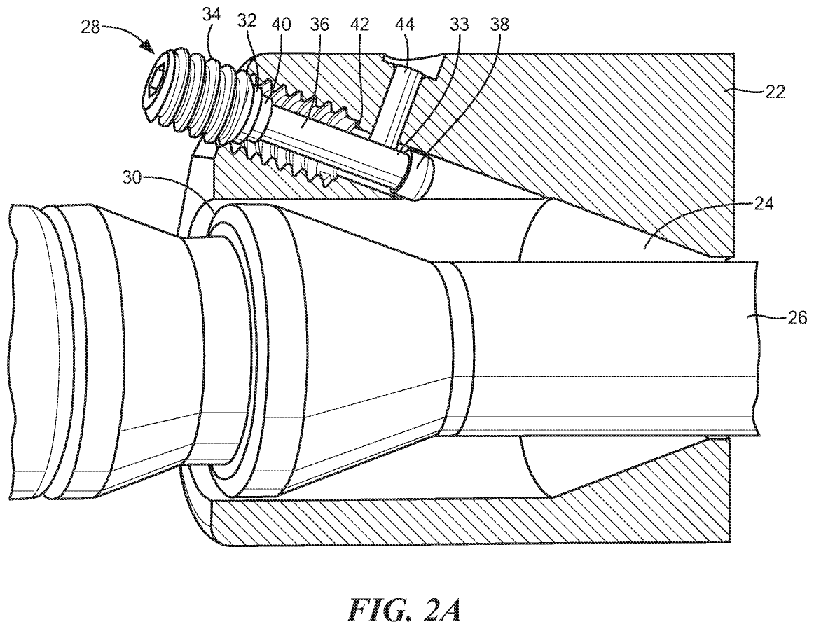

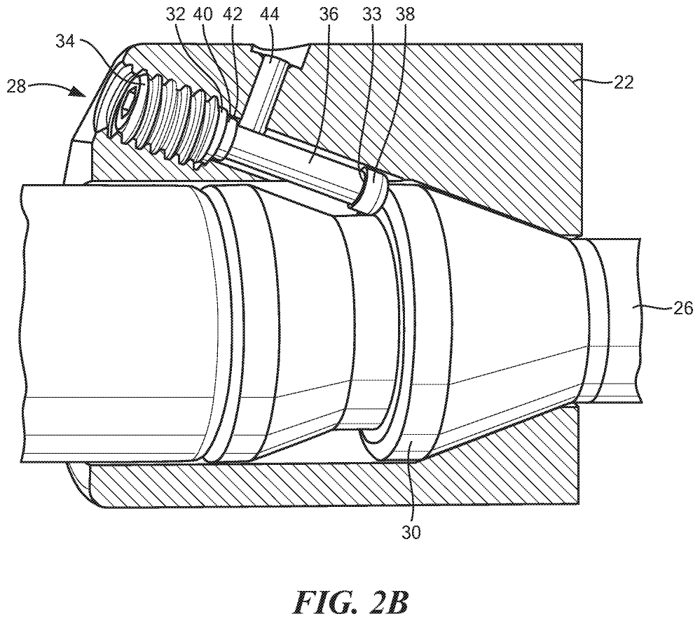

Referring now to FIGS. 2A-2B, the controller 10 may include a header or housing 22 defining a one or more header or housing bores 24 sized and configured to received one or more corresponding connectors 26 connected to the one or more implanted conductors 20. In the configuration shown in FIG. 2A, the housing bores 24 taper as they extend distally. A set screw 28 is releasably coupled to the housing 22 and is disposed at an oblique angle with respect to the housing 22. The set screw 28 is configured to engage at least a portion of the connector 26 when the set screw is fully inserted within the housing 22 to lock the connector 26 within the housing 22. In one configuration, the set screw is disposed at a 30-degree angle with respect to the housing 22 although any oblique angle is contemplated. In one configuration, the set screw 28 is configured to engage a flange 30 of a distal end of the connector 26 when fully inserted within the housing 22. For example, the connector 26 may include a bushing including the flange 30 which provides a contact point for engaging the set screw 28 to lock the connector 26 to the housing 22.

Continuing to refer to FIGS. 2A-2B, the set screw 28 includes a proximal portion 32 and a distal portion 33. The proximal portion 32 includes a plurality of threads 34 and the distal portion includes an elongate rod 36. In one configuration, the elongate rod 36 defines a diameter less than a diameter of the plurality of threads 34. The elongate rod 36 includes a proximal end and a distal end, and wherein the distal end includes a stop shoulder 38 and is further configured to engage the flange 30 when the set screw 28 is fully inserted within the housing 22. The proximal end of the elongate rod 36 includes a shoulder 40. The shoulder 40, which may be a chamfered, is configured to engaged a corresponding depth stop 42 defined by the housing 22. That is, the set screw 28 may be advanced within the housing 22 until the shoulder 40 contacts the depth stop 42. At this point, the distal end of the elongate rod 36 contacts the flange 30. The distal end of the elongate rod 36 may be atraumatic in shape as to not damage the connector 26. A retainer pin 44 is retained within the housing 22. The retainer pin 44 is configured to prevent the set screw 28 from being removed when the set screw 28 is fully retracted. In one configuration, the retainer pin 44 is disposed at an oblique angle with respect to the housing 22 and may be orthogonal with respect to the set screw 28. In particular, as the set screw 28 is retracted, the stop shoulder 38 contacts the retainer pin 44, which when fully inserted within the housing 22, prevents the set screw 28 from being retracted.

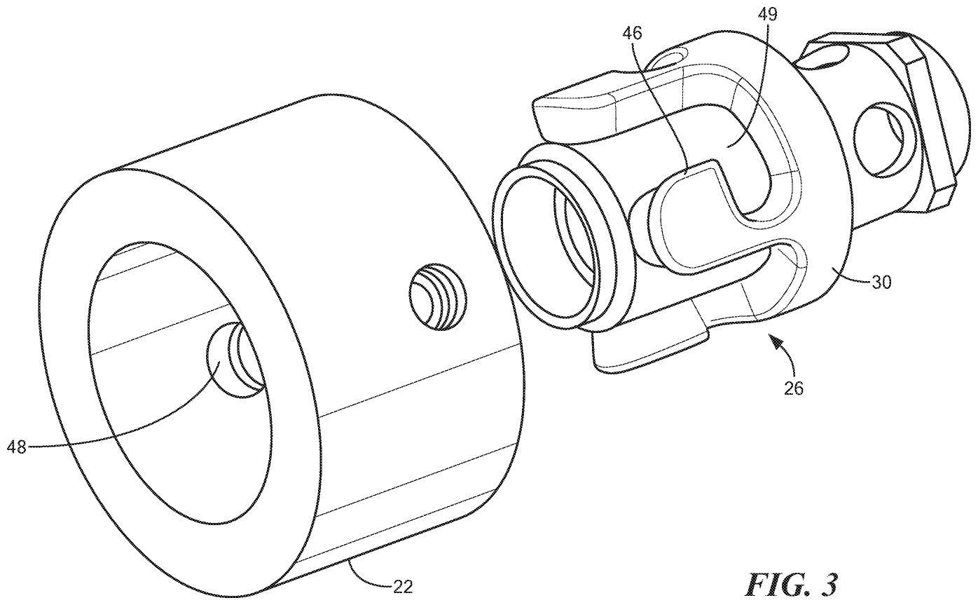

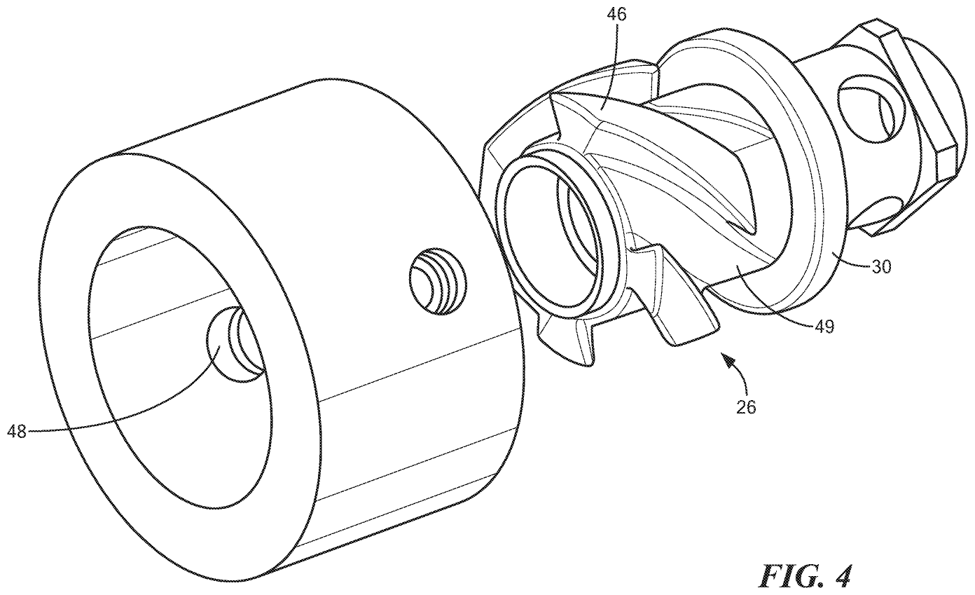

Referring now to FIGS. 3 and 4, in another configuration, the connector 26 is configured to slidably engage with the housing 22 to lock the connector 26 to the housing 22. For example, the connector may include the flange 30 as shown in FIGS. 2A-B however the bushing includes a plurality of guides 46 configured to guide a corresponding protuberance 48 on the housing 22 to lock with the flange 30. In the configuration shown in FIG. 3, each guide 46 defines a corresponding slot 49 sized to receive the protuberance and the slot 48 defines an L-shape with the guide 46 to receive and lock the protuberance with a push and a rotation of the connector 26 within the housing 22. In the configuration shown in FIG. 4, the slot 49 defines a Z-shape with the corresponding guide 46 such that the connector may be twist-locked with the housing 22 when the protuberance 48 is slid within the slot 49.

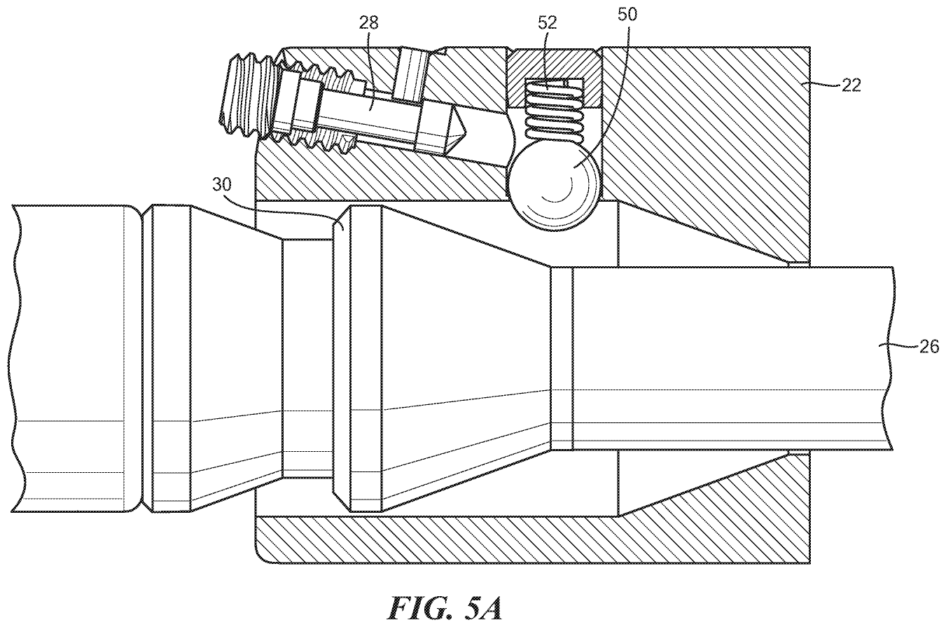

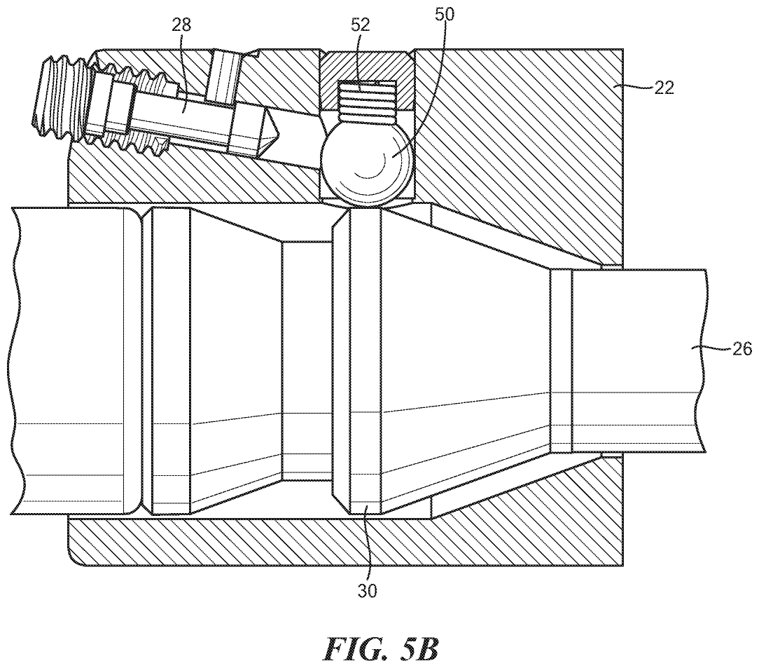

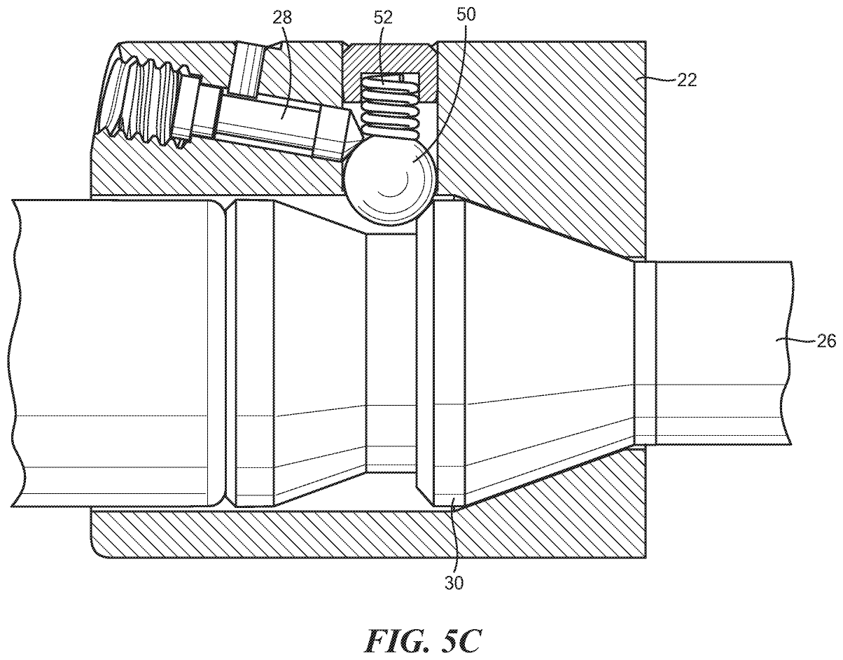

Referring now to FIG. 5A-5C, in yet another configuration, the connector 26 is configured to engage an atraumatic contact element 50, which is further engaged to a biasing element 52, for example, a spring. In one configuration, the atraumatic contact element 50 is a sphere that floats in a position substantially orthogonal to the connector 26 (FIG. 5A). As the connector 26 is advanced into the housing 22, the flange 30 pushes up on the atraumatic contact element 50 (FIG. 5B) and once the flange is advanced passed the atraumatic contact element 50, the atraumatic contact element 50 is biased downward against the opposite side of the flange 30 (FIG. 5C). This provides the clinician with tactile feedback that the connector 26 is properly positioned within the housing 22. Once the atraumatic contact element 50 is positioned on the opposite side of the flange 30, the set-screw 28 is advanced to contact the atraumatic contact element 50 and lock the connector 26 within the housing 22.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A locking mechanism for a connector to an implantable controller, comprising:
a housing of the implantable controller defining a housing bore sized to receive the connector; and
a set screw releasably coupled to the housing and disposed at an oblique angle with respect to the housing, the set screw being configured to engage at least a portion of the connector when the set screw is fully inserted within the housing to lock the connector within the housing.

2. The mechanism of claim 1, wherein the set screw is configured to engage a flange of a distal end of the connector when fully inserted within the housing.

3. The mechanism of claim 2, wherein the housing bore tapers in diameter as it extends distally.

4. The mechanism of claim 3, wherein the set screw includes a proximal portion and a distal portion, and wherein the proximal portion includes a plurality of threads and the distal portion includes an elongate rod.

5. The mechanism of claim 4, wherein the elongate rod includes a proximal end and a distal end, and wherein the distal end includes a stop shoulder, and wherein the distal end is configured to engage the flange when the set screw is fully inserted within the housing.

6. The mechanism of claim 5, wherein the proximal end of the elongate rod includes a shoulder, and wherein the shoulder is configured to engaged a corresponding depth stop defined by the housing.

7. The mechanism of claim 6, wherein the stop shoulder is atraumatic in shape.

8. The mechanism of claim 1, further including a retainer pin retained within the housing, the retainer pin being configured to prevent the set screw from being removed when the set screw is fully retracted.

9. The mechanism of claim 8, wherein the retainer pin is disposed at an angle orthogonal to the set screw.

10. The mechanism of claim 9, wherein the retainer pin is disposed at an oblique angle with respect to the housing.

11. The mechanism of claim 1, further comprising an atraumatic locking element, wherein the set screw is configured to engaged to an engage the atraumatic locking element, and wherein the atraumatic locking element is pushed into contact with the connector when then set screw is fully inserted.

12. The mechanism of claim 11, wherein the atraumatic locking element is a sphere.

13. A locking mechanism for a connector to an implantable controller, comprising:
a housing of the implantable controller defining a housing bore sized to receive the connector, the housing bore defining a diameter that tapers as it extends distally;
a set screw releasably coupled to the housing and disposed at an oblique angle with respect to the housing, the set screw being configured to engage a flange of the connector when the set screw is fully inserted within the housing to lock the connector within the housing; and
a retainer pin releasably retained within the housing, the retainer pin being configured to prevent the set screw from being removed when the set screw is fully retracted.

14. The mechanism of claim 13, wherein the set screw includes a proximal portion and a distal portion, and wherein the proximal portion includes a plurality of threads and the distal portion includes an elongate rod.

15. A locking mechanism for a connector to an implantable controller, comprising:
a housing of the implantable controller defining a housing bore sized to receive the connector, the housing bore defining a diameter that tapers as it extends distally;
a set screw releasably coupled to the housing and disposed at an oblique angle with respect to the housing, the set screw being configured to engage a flange of the connector when the set screw is fully inserted within the housing to lock the connector within the housing, the set screw includes a proximal portion and a distal portion, and wherein the proximal portion includes a plurality of threads and the distal portion includes an elongate rod, the elongate rod includes a proximal end and a distal end, and wherein the distal end includes an atraumatic stop shoulder, and wherein the stop shoulder is configured to engage the flange when the set screw is fully inserted within the housing, the proximal end of the elongate rod further includes a chamfered shoulder, and the chamfered shoulder is configured to engaged a corresponding depth stop defined by the housing; and
a retainer pin releasably retained within the housing and disposed at an oblique angle with respect to the housing and orthogonally disposed with respect to the set screw, the retainer pin being configured to prevent the set screw from being removed when the set screw is fully retracted.

16. The mechanism of claim 14, wherein the elongate rod includes a proximal end and a distal end, and wherein the distal end includes a stop shoulder, and wherein the stop shoulder is configured to engage the flange when the set screw is fully inserted within the housing.

17. The mechanism of claim 14, wherein the proximal end of the elongate rod includes a chamfered shoulder, and wherein the chamfered shoulder is configured to engage a corresponding depth stop defined by the housing.

18. The mechanism of claim 13, wherein the retainer pin is disposed at an oblique angle with respect to the housing.

19. The mechanism of claim 13, wherein the retainer pin is disposed at an angle orthogonal to the set screw.
